(19) United States
(12) Patent Application Publication
(10) Pub. No.: US 2024/0166986 A1
(43) Pub. Date: May 23, 2024

(54) Recombinant Escherichia Coli for Producing L-tyrosine and Application Thereof

(71) Applicant: Jiangnan University, Wuxi (CN)

(72) Inventors: Jingwen Zhou, Wuxi (CN); Jian Chen, Wuxi (CN); Jurong Ping, Wuxi (CN); Weizhu Zeng, Wuxi (CN)

(21) Appl. No.: 18/428,104

(22) Filed: Jan. 31, 2024

(30) Foreign Application Priority Data
Feb. 21, 2023 (CN) 2023101438690

(51) Int. Cl. C12N 1/20; C12N 9/10; C12N 9/12; C12N 15/70; C12P 13/22; C12R 1/19

(52) U.S. Cl. CPC C12N 1/20; C12N 9/1022; C12N 9/1294; C12N 15/70; C12P 13/22; C12R 2001/19; C12Y 202/01001; C12Y 207/09002

(57) ABSTRACT

Disclosed is recombinant Escherichia coli for producing L-tyrosine and application thereof, and belongs to the technical fields of genetic engineering and bioengineering. According to the present disclosure, genes aroP and tyrP are knocked out, expresses the endogenous gene yddG of E. coli, then heterologously expresses fpk from Bifidobacterium adolescentis, expresses the endogenous genes ppsA and tktA of E. coli, and then expresses aroG^{fbr} and tyrA^{fbr}. Knocking out tyrR, trpE and pheA, so that the synthesis flux of L-tyrosine is increased. Finally, an endogenous gene poxB is knocked out to realize stable fermentation performance at high glucose concentration.

### REFERENCE TO SEQUENCE LISTING

The instant application contains a Sequence Listing in XML format as a file named “YGHY-2023-39-SEQ.xml”, created on Jan. 16, 2024, of 108 kB in size, and which is hereby incorporated by reference in its entirety.

### TECHNICAL FIELD

The present disclosure relates to recombinant Escherichia Coli for producing L-tyrosine and application thereof, and belongs to the technical fields of genetic engineering and bioengineering.

### BACKGROUND

L-tyrosine (Tyr), as an essential amino acid, is an aromatic amino acid among 20 amino acids forming proteins, which has been widely used in food, feed, medicine and other fields. The L-tyrosine can promote synthesis of catecholamines, thyroid hormones and melanin in human bodies, which has an important effect on development and metabolism of humans and animals. In medicine, the L-tyrosine is used as a main raw material for synthesis of many drugs such as thyroid hormones, epinephrine and levodopa.

At present, traditional production methods for the L-tyrosine include a protein hydrolysis method, a chemical synthesis method, an enzyme method and a microbial fermentation method. According to the protein hydrolysis method, also known as an extraction method, natural protein resources, such as casein, swine blood meal, animal hoofs, horns, hairs and other raw materials are used for separating and extracting the L-tyrosine through hydrolysis, concentration, crystallization, decolorization and other steps. According to an enzyme conversion method, phenol, an ammonia salt and pyruvic acid are used as precursors and converted by tyrosinase. However, the enzyme is easily inactivated, and reaction conditions are strict, so that the enzyme method is not used for industrial preparation of the L-tyrosine in a large scale. According to the chemical synthesis method, racemic DL-tyrosine is synthesized by hydroxylation of L-phenylalanine or by condensation of p-hydroxyamphetamine and hydantoin, alkali hydrolysis, ammonia conversion and other steps, and the L-tyrosine needs to be further separated, resulting in that the process is complicated, and the efficiency is low. According to the microbial fermentation method, biomass raw materials are used to achieve de novo synthesis of tyrosine, so that the production cost is greatly reduced. Compared with the protein hydrolysis method and an enzyme hydrolysis method, the microbial fermentation method has the advantages of short cycle, high conversion rate, simple separation and purification steps and the like. Since the yield of existing tyrosine producing bacteria is low, it is difficult to achieve industrial production in a large scale. It is urgent to construct a recombinant strain capable of producing L-tyrosine efficiently and to establish a microbial fermentation method with higher yield.

### SUMMARY

In order to solve the above technical problems, the present disclosure provides recombinant Escherichia coli for synthesizing L-tyrosine. E. coli, as an original strain, is subjected to at least one of the following improvements:
(1) knocking out endogenous aroP and tyrP to block the transport of L-tyrosine from the outside of cells to the inside of cells of E. coli;
(2) overexpressing endogenous yddG to improve the ability of E. coli to transport L-tyrosine from the outside of cells to the inside of cells;
(3) expressing phosphoketolase (fpk) from Bifidobacterium adolescentis and endogenous genes ppsA and tktA of E. coli to effectively guide a carbon metabolism flow of glucose to synthesis of L-tyrosine;
(4) expressing a mutant aroG^{fbr} of aroG from E. coli, where the mutant is a mutant relieving feedback inhibition of an aromatic amino acid on 3-deoxy-D-arabino-heptulosonate-7-phosphate synthase;
(5) expressing a mutant tyrA^{fbr} of tyrA from E. coli, where the mutant is used for relieving the effect of excessive accumulation of L-tyrosine on chorismate mutase and prephenate dehydrogenase, so that the synthesis flux of L-tyrosine is effectively increased;
(6) knocking out endogenous pheA and trpE to block some branches of a synthetic route of a shikimic acid pathway, so that more metabolism flows are used for synthesizing L-tyrosine;
(7) expressing endogenous genes ppsA and tktA of E. coli to increase precursor supply of a shikimic acid pathway and increase the synthesis flux of L-tyrosine; and
(8) knocking out a gene poxB to reduce the accumulation of acetic acid in a fermentation process and realize fermentation at high glucose concentration.

The present disclosure provides recombinant Escherichia coli for synthesizing L-tyrosine efficiently. The recombinant E. coli is obtained by using E. coli as an original strain and subjecting the strain to any one of the following gene editing processes (a)-(d):
(a) knocking out a gene pheA encoding and fusing chorismate mutase/prephenate dehydratase, a gene trpE encoding an anthranilate synthetase subunit TrpE, a gene tyrR encoding a DNA binding transcription double regulator TyrR, a gene aroG^{fbr} freely expressing and encoding 3-deoxy-D-arabino-heptulosonate-7-phosphate synthase and a gene tyrA^{fbr} encoding chorismate mutase and prephenate dehydrogenase on an E. coli genome;
(b) knocking out a gene pheA encoding and fusing chorismate mutase/prephenate dehydratase, a gene trpE encoding an anthranilate synthetase subunit TrpE, a gene tyrR encoding a DNA binding transcription double regulator TyrR, a gene aroG^{fbr} freely expressing and encoding 3-deoxy-D-arabino-heptulosonate-7-phosphate synthase, a gene tyrA^{fbr} encoding chorismate mutase and prephenate dehydrogenase and a gene fpk encoding phosphoketolase on an E. coli genome;
(c) knocking out a gene pheA encoding and fusing chorismate mutase/prephenate dehydratase, a gene trpE encoding an anthranilate synthetase subunit TrpE, a gene tyrR encoding a DNA binding transcription double regulator TyrR, a gene poxB encoding pyruvate oxidase, a gene aroG^{fbr} freely expressing and encoding 3-deoxy-D-arabino-heptulosonate-7-phosphate synthase, a gene tyrA^{fbr} encoding chorismate mutase and prephenate dehydrogenase and a gene fpk encoding phosphoketolase on an E. coli genome;
(d) knocking out a gene pheA encoding and fusing chorismate mutase/prephenate dehydratase, a gene trpE encoding an anthranilate synthetase subunit TrpE, a gene tyrR encoding a DNA binding transcription double regulator TyrR, a gene poxB encoding pyruvate oxidase, a gene aroP encoding permease of an aromatic amino acid transporter AroP, a gene tyrP encoding a tyrosine and H (+) sympoter, a gene aroG^{fbr} freely expressing and encoding 3-deoxy-D-arabino-heptulosonate-7-phosphate synthase, a gene tyrA^{fbr} encoding chorismate mutase and prephenate dehydrogenase, a gene fpk encoding phosphoketolase and a gene yddG encoding an amino acid exportin YddG on an E. coli genome.

In one embodiment, the recombinant E. coli integrates a gene ppsA expressing phosphoenolpyruvate synthetase and a gene tktA encoding transketolase 1.

In one embodiment, the gene ppsA is integrated at a site ykgh-betA on the E. coli genome.

In one embodiment, the gene tktA is integrated at a site dadx-cvra on the E. coli genome.

In one embodiment, the gene ppsA and the gene tktA are initially expressed by a promoter PJ₂₃₁₁₁₉.

In one embodiment, a heat induced expression vector pAP-B03 is used as an expression plasmid of the E. coli.

In one embodiment, the gene pheA has a nucleotide sequence set forth in SEQ ID NO:1.

In one embodiment, the gene trpE has a nucleotide sequence set forth in SEQ ID NO:2.

In one embodiment, the gene aroG^{fbr} has a nucleotide sequence set forth in SEQ ID NO:3.

In one embodiment, the gene tyrA^{fbr} has a nucleotide sequence set forth in SEQ ID NO:4.

In one embodiment, the gene tyrR has a nucleotide sequence set forth in SEQ ID NO:5.

In one embodiment, the gene ppsA has a nucleotide sequence set forth in SEQ ID NO:6.

In one embodiment, the gene tktA has a nucleotide sequence set forth in SEQ ID NO:7.

In one embodiment, the gene fpk has a nucleotide sequence set forth in SEQ ID NO:8.

In one embodiment, the gene poxB has a nucleotide sequence set forth in SEQ ID NO:9.

In one embodiment, the gene aroP has a nucleotide sequence set forth in SEQ ID NO:10.

In one embodiment, the gene tyrP has a nucleotide sequence set forth in SEQ ID NO:11.

In one embodiment, the gene yddG has a nucleotide sequence set forth in SEQ ID NO:12.

In one embodiment, the promoter PJ₂₃₁₁₁₉ has a nucleotide sequence set forth in SEQ ID NO:101.

In one embodiment, the heat induced expression vector pAP-B03 plasmid is used for freely expressing the aroG^{fbr}, the fpk, the yddG and the tyrA^{fbr}.

In one embodiment, the heat induced expression vector pAP-B03 plasmid is recorded in the document “Zhou, H., Liao, X., Wang, T., Du, G., Chen, J., 2010. Enhanced L-phenylalanine biosynthesis by co-expression of pheA^{fbr} and aroF^{wt}. Bioresource Technology. 101(11): 4151-4156.”.

In one embodiment, E. coli K12, E. coli BL21, E. coli DH5α, E. coli JM109 or E. coli HG is used as the original strain.

In one embodiment, the strain E. coli HG is a strain WSH-Z06 (pAP-B03) recorded in the document “Zhou, H., Liao, X., Wang, T., Du, G., Chen, J., 2010. Enhanced L-phenylalanine biosynthesis by co-expression of pheA^{fbr} and aroF^{wt}. Bioresource Technology. 101(11): 4151-4156.”.

The present disclosure provides a method for producing L-tyrosine. The method includes using the recombinant E. coli to produce the L-tyrosine by fermentation.

In one embodiment, the recombinant E. coli is inoculated into a fermentation system, cultured at 32-34° C. for 3-12 h and fermented at 200-220 rpm at 36-40° C. for 48-60 h.

In one embodiment, the fermentation system includes 30-40 g/L glucose, 3-7 g/L (NH₄)₂SO₄, 1-5 g/L KH₂PO₄, 1-5 g/L MgSO₄·7H₂O, 1-2 g/L sodium citrate, 0.5-1.5 g/L NaCl, 0.05-0.1 g/L vitamin B₁, 0.1-0.12 g/L FeSO₄·7H₂O, 1-3 g/L yeast powder, 2-6 g/L peptone and 1-2 mL/L trace element nutrient solution (TES).

In one embodiment, the TES includes the following components: 2.0 g/L Al₂(SO₄)₃·18H₂O, 0.75 g/L CoSO₄·7H₂O, 2.5 g/L CuSO₄·5H₂O, 0.5 g/L H₃BO₃, 24 g/L MnSO₄·H₂O, 2.5 g/L NiSO₄·6H₂O and 15 g/L ZnSO₄·7H₂O.

The present disclosure provides application of the recombinant E. coli in production of L-tyrosine or a product containing L-tyrosine.

### The Present Disclosure has the Following Beneficial Effects

1. According to the present disclosure, with E. coli as a host, in order to increase the carbon flux of a synthetic route for synthesis of tyrosine, a gene pheA encoding chorismate mutase-prephenate dehydratase (CM-PDT) in the E. coli is knocked out by a CRISPR/Cas9 gene editing method, aroG^{fbr}, tyrA^{fbr}, tktA and ppsA are freely expressed by a heat induced plasmid pAP-B03, a gene trpE encoding an anthranilate synthetase subunit trpE is further knocked out to block the synthesis of tryptophan, and a gene tyrR is knocked out to relieve a repression effect of a protein TyrR on key enzymes of a shikimic acid pathway. After fermentation for 48 h, 5.6 g/L tyrosine is accumulated in a shake flask.
2. In order to further improve the utilization of glucose, a gene fpk encoding phosphoketolase from Bifidobacterium adolescentis is heterologously expressed to directionally guide glucose to a shikimic acid pathway so as to increase precursor supply of the shikimic acid pathway, genes tktA and ppsA are integrated and expressed on an E. coli genome, and a strong promoter PJ₂₃₁₁₁₉ is used to initiate gene expression. After fermentation for 48 h, 6.0 g/L L-tyrosine is accumulated in a shake flask.
3. An acetic acid pathway of E. coli is modified, and a gene poxB encoding pyruvate oxidase (PoxB) on the E. coli genome is knocked out to effectively reduce the production of acetic acid, so that the content of the L-tyrosine is increased to 6.2 g/L.
4. Genes aroP and tyrP are knocked out, an aromatic amino acid transport system of E. coli is modified by freely expressing an endogenous gene yddG of E. coli, then a gene fpk encoding phosphoketolase is freely expressed, the genes ppsA and tktA are integrated and expressed to increase precursor supply of the shikimic acid pathway, and then feedback inhibition of 3-deoxy-D-arabino-heptulosonate-7-phosphate synthase, chorismate mutase and prephenate dehydrogenase is relieved by expressing anti-feedback genes of aroG^{fbr} and tyrA^{fbr}, so that the carbon flux of the shikimic acid pathway is increased. A repression effect of a protein TyrR on key enzymes of the shikimic acid pathway is relieved by knocking out a gene tyrR, and partial side reactions of the shikimic acid pathway are blocked by knocking out genes trpE and pheA, so that the synthesis flux of tyrosine is increased. Finally, an endogenous gene poxB is knocked out to realize transformation of an acetic acid pathway, so that stable fermentation performance at high glucose concentration is realized. When an engineered strain of E. coli obtained by the method of the present disclosure is subjected to induced fermentation for 55 h, the content of L-tyrosine in a fermentation liquid is as high as 80.5 g/L, and the production intensity reaches 1.46 g/L/h, so that a new idea is provided for industrial production of L-tyrosine.

### BRIEF DESCRIPTION OF FIGURES

FIG. 1 is a diagram showing synthesis of L-tyrosine in E. coli.

FIG. 2 is a diagram showing yield of L-tyrosine obtained by feed-batch fermentation of E. coli in a 5 L fermentation tank.

### DETAILED DESCRIPTION

(I) Culture Media

A seed culture medium (LB) includes: 10 g/L peptone, 5 g/L a yeast extract and 5 g/L sodium chloride; and 2% (mass fraction) agar powder was added into a solid culture medium.

A fermentation culture medium (1 L) includes: 35 g of glucose, 5 g of (NH₄)₂SO₄, 3 g of K₂HPO₄·3H₂O, 3 g of MgSO₄·7H₂O, 1.5 g of sodium citrate, 1 g of NaCl, 0.075 g of vitamin B₁, 0.1125 g of FeSO₄·7H₂O, 2 g of yeast powder, 4 g of peptone and 1.5 mL of a trace element nutrient solution (TES), and appropriate amounts of antibiotics were added as required. 12 g of calcium carbonate was added into a conical flask to control the pH value; and the TES includes: 2.0 g/L Al₂(SO₄)₃·18H₂O, 0.75 g/L CoSO₄·7H₂O, 2.5 g/L CuSO₄·5H₂O, 0.5 g/L H₃BO₃, 24 g/L MnSO₄·H₂O, 2.5 g/L NiSO₄·6H₂O and 15 g/L ZnSO₄·7H₂O.

(II) PCR Reaction System and Amplification Conditions

1 μL (10 μM) of a forward primer, 1 μL (10 μM) of a reverse primer, 10-50 ng of template DNA and 25 μL of 2×Phanta Max Master Mix, and double distilled water added to 50 μL. Amplification conditions include: pre-denaturation at 95° C. for 3 min, followed by 30 cycles (at 95° C. for 15 s, at 55° C. for 15 s, and at 72° C. for 15 s) and continuous extension at 72° C. for 5 min.

(III) Preparation of E. coli Competent Cells

E. coli K12 in a glycerol tube was streaked on a corresponding LB plate and cultured overnight at 37° C. (for about 12 h). 12 h later, monoclone is picked, inoculated into a 50 mL shake flask containing 5 mL of an LB culture medium and cultured at 220 rpm at 37° C. until an OD₆₀₀ value was 0.6-0.8. A bacterial solution was transferred to a 50 mL centrifuge tube, placed on ice for about 15 min and centrifuged at 4,000 rpm at 4° C. for 5 min to remove a supernatant. 5 mL of a solution A was added for resuspension, and centrifugation was performed at 4,000 rpm at 4° C. for 5 min to remove a supernatant. Then, 5 mL of a solution B was added to resuspend the bacteria, and a resulting product was packaged in 100 μL/part and store at −80° C.

(IV) Transformation of E. coli

E. coli competent cells were thawed on ice. 10 μL of a recombinant product (50 ng of plasmid) was added into 100 μL of the competent cells, evenly mixed by flicking, and subjected to standing on ice for 30 min. A resulting mixture was subjected to heat shock in a water bath pot at 42° C. for 45 s, followed by standing on ice for 2 min. 1 mL of an LB culture medium was added, and the bacteria were shaken at 220 rpm at 37° C. for 60 min. Then, centrifugation was performed at 4,500 rpm for 2 min to remove a supernatant. The bacteria were resuspended with the remaining culture medium and then coated on a resistant plate.

(V) Determination of L-Tyrosine by High Performance Liquid Chromatography (HPLC)

After completion of fermentation, 1 mL of a fermentation liquid was taken, diluted to an appropriate multiple with 3 M hydrochloric acid, violently shaken and uniformly mixed, followed by centrifugation at 14,000 rpm for 10 min. A supernatant was taken and filtered with a 0.22 μm inorganic filter membrane, and a product was detected by a high performance liquid chromatograph LC-20A of Shimadzu. A Thermo Fisher C18 chromatographic column (4.6 mm×250 mm, 5 μm) was used for chromatographic separation; the temperature of a column oven was set to 30° C.; the injection volume was 10 μL; mobile phases were as follows: phase A: 0.1 M sodium acetate (the pH was adjusted to 4.5 with glacial acetic acid), and phase B: pure methanol; the total flow rate was 1 mL/min; the volume percentage was 90% and 10%, respectively; and the wavelength of a detector was 280 nm.

(VI) Plasmids

A plasmid pAP-B03 involved in the following examples is recorded in the document “Zhou, H., Liao, X., Wang, T., Du, G., Chen, J., 2010. Enhanced L-phenylalanine biosynthesis by co-expression of pheA^{fbr} and aroF^{wt}. Bioresource Technology. 101(11): 4151-4156.”. Plasmids pCas and p-Target involved in the following examples are recorded in the document “Jiang, Y., Chen, B., Duan, C., Sun, B., Yang, J., Yang, S., 2015. Multigene editing in the Escherichia coli genome via the CRISPR-Cas9 system. Applied and Environmental Microbiology. 81(7): 2506-2514.”. A recombinant plasmid pCDF-aroG^{fbr}-tyrA^{fbr} involved in the following examples is recorded in the document “Wu, J., Zhou, T., Du, G., Zhou, J., Chen, J., 2014. Modular optimization of heterologous pathways for de novo synthesis of (2S)-naringenin in Escherichia coli. PloS One. 9(7): 1-9.”. A strain E. coli HG involved in the following examples is a strain WSH-Z06 (pAP-B03) recorded in the document “Zhou, H., Liao, X., Wang, T., Du, G., Chen, J., 2010. Enhanced L-phenylalanine biosynthesis by co-expression of pheA^{fbr} and aroF^{wt}. Bioresource Technology. 101(11): 4151-4156.”, which is named as E. coli HG in the present disclosure.

(VII) Information of Strains as Shown in Table 1

TABLE 1
Strains and genes involved in the present disclosure

| Strain name | Genotype |
|---|---|
| E. coli HG0 | E. coli HG eliminate pAP-pheA^{fbr}-aroF^{fbr} |
| E. coli HGA | E. coli HG0 ΔpheA containing pAP-aroG^{fbr}-tyrA^{fbr}-ppsA-tktA |
| E. coli HGB | E. coli HG0 ΔpheAΔtrpE containing pAP-aroG^{fbr}-tyrA^{fbr}-ppsA-tktA |
| E. coli HGC | E. coli HG0 ΔpheAΔtrpEΔtyrR containing pAP-aroG^{fbr}-tyrA^{fbr}-ppsA-tktA |
| E. coli HGD0 | E. coli HG0 ΔpheAΔtrpEΔtyrR dadx-cvra::tktA |
| E. coli HGE | E. coli HGD0 ykgh-betA::ppsA containing pAP-aroG^{fbr}-tyrA^{fbr}-fpk |
| E. coli HGF | E. coli HGE ΔpoxB containing pAP-aroG^{fbr}-tyrA^{fbr}-fpk |
| E. coli HGG | E. coli HGF0 ΔaroP containing pAP-aroG^{fbr}-tyrA^{fbr}-fpk |
| E. coli HGH0 | E. coli HGF0 ΔaroPΔtyrP |
| E. coli HGH | E. coli HGF0 ΔaroPΔtyrP containing pAP-aroG^{fbr}-yddG-tyrA^{fbr}-fpk |

### Example 1: Construction of Recombinant E. coli for Synthesis of L-Tyrosine

(1) Preparation of an Engineered Strain HGA0

E. coli HG stored in a laboratory was continuously subjected to passage culture at 42° C. for removing a plasmid to obtain a plasmid-free strain HG0. A plasmid pCas was transformed into chemically transformed competent cells of the E. coli HG0; monoclone obtained by transformation is picked into a 4 mL of an LB culture medium containing 50 μg/mL kanamycin and cultured at 30° C. for 12 h; then a bacterial solution with a volume ratio of 2% was inoculated into 50 mL of an LB culture medium; and kanamycin with a final concentration of 50 μg/mL and a 10 mM arabinose solution were added. A mixed solution was cultured at 220 rpm at 30° C. for 4-6 h, and when the optical density (OD) value was 0.6, the bacterial solution was transferred to a 50 mL centrifuge tube and subjected to standing on ice for 15 min. Centrifugation was performed at 4,000 rpm at 4° C. for 10 min to remove a supernatant, and 10 mL of 10% glycerol was added for resuspension; the operation was repeated twice, and a resulting product was packaged at 100 μL/part and stored at −80° C. to obtain electrically transformed competent cells of E. coli HG0 containing a plasmid pCas, named as E. coli HG0-pCas.

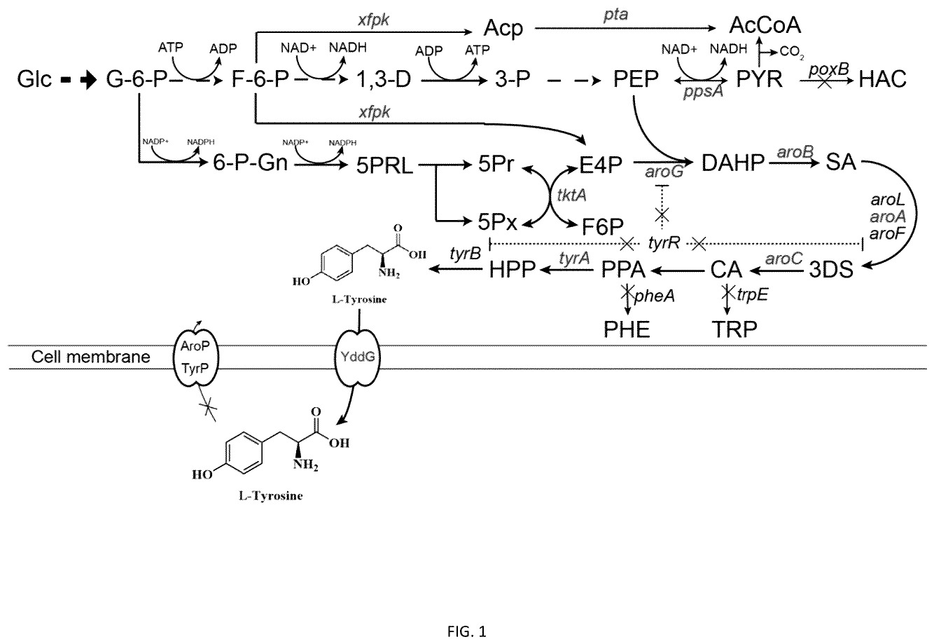

The E. Coli HG0 was selected as an original strain for production of L-tyrosine by fermentation. First, according to a synthetic route of L-tyrosine shown in FIG. 1, in order to increase the carbon flux of a synthetic route for synthesis of tyrosine, a gene pheA encoding chorismate mutase-prephenate dehydratase (CM-PDT) in E. coli was knocked out by a CRISPR/Cas9 gene editing method. With an E. coli K12 genome as a template, an upstream homologous arm U1 and a downstream homologous arm D1 of the gene pheA were amplified with primer pairs F11/R11 and F12/R12, respectively, and fragments were purified. With purified fragments U1 and D1 as templates, a knockout box UD1 was obtained by amplification with a primer pair F11/R12, and fragments were purified. In order to obtain pTarget-pheA for knocking out the pheA, amplification was performed with a primer pair F13/R13 with p-Target stored in a laboratory as a template, and fragments were purified. A purified fragment pTarget-pheA was transformed into E. coli JM109, and a plasmid was extracted and sequenced for verification to obtain a correct recombinant vector pTarget-pheA.

400 ng of the recombinant vector pTarget-pheA and 1,200 ng of the knockout box UD1 were added into the electrically transformed competent cells of E. coli HG0-pCas, and a mixture was subjected to standing on ice for 10 min, transferred into a 1 mm electroporation cuvette precooled for 10 min and then subjected to electric shock at a voltage of 1.8 kv. After completion of the electric shock, 1 mL of an LB liquid culture medium was added and cultured at 30° C. for 1.5 h. Bacterial colonies were subjected to PCR verification with a primer pair F14/R14, and the verified monoclone loses the pTarget-pheA and the pCas9 to obtain an engineered strain of E. coli HGA0.

(2) Preparation of Overexpression Plasmids pAP-aroG^{fbr}-tyrA^{fbr} and pAP-aroG^{fbr}-tyrA^{fbr}-ppsA-tktA and an Engineered Strain HGA

A heat induced plasmid framework was obtained from a plasmid pAP-B03 with a primer pair F113/R113, including a kanamycin gene, a promoter P_{R}P_{L} (obtained with a primer pair F116/R116) and a replicator p15A. Genes aroG^{fbr} and tyrA^{fbr} were obtained from a plasmid pCDF-aroG^{fbr}-tyrA^{fbr} with primer pairs F114/R114 and F117/R117, respectively. A gene tktA and a gene ppsA were obtained from an E. coli genome by amplification with primer pairs F115/R115 and F118/R118, respectively. The obtained heat induced plasmid framework was assembled with the target genes aroG^{fbr}, tyrA^{fbr}, tktA and ppsA by a Gibson assembly method to obtain plasmids pAP-aroG^{fbr}-tyrA^{fbr} and pAP-aroG^{fbr}-tyrA^{fbr}-ppsA-tktA.

The recombinant vector pAP-aroG^{fbr}-tyrA^{fbr}-ppsA-tktA was transformed into the E. coli HGA0 to obtain an engineered strain HGA.

(3) Preparation of Engineered Strains HGB0 and HGB

With the engineered strain HGA0 as an original strain, electrically transformed competent cells HGA0-pCas of HGA0 containing a plasmid pCas were constructed by the same method. A gene trpE was knocked out by the same method to block the synthesis of tryptophan. With an E. coli K12 genome as a template, upstream and downstream homologous arms of the trpE were amplified with primer pairs F15/R15 and F16/R16, respectively, and a knockout box UD2 was obtained by amplification with a primer pair F15/R16. p-Target was amplified with a primer pair F17/R17 to prepare a recombinant vector pTarget-trpE. The knockout box UD2 and the recombinant vector pTarget-trpE were electrically transformed into the HGA0-pCas. Bacterial colonies were subjected to PCR verification with a primer pair F18/R18, and the verified monoclone loses the pTarget-trpE and the pCas9 to obtain an engineered strain HGB0.

The recombinant vector pAP-aroG^{fbr}-tyrA^{fbr}-ppsA-tktA was transformed into the E. coli HGB0 to obtain an engineered strain HGB.

(4) Preparation of Engineered Strains HGC0 and HGC

With the engineered strain HGB0 as an original strain, electrically transformed competent cells HGB0-pCas of HGB0 containing a plasmid pCas were constructed by the same method. With an E. coli K12 genome as a template, a gene tyrR was knocked out by the same method to relieve a repression effect of accumulation of amino acids on key enzymes of a shikimic acid pathway, upstream and downstream homologous arms of the tyrR were amplified with primer pairs F19/R19 and F110/R110, respectively, and a knockout box UD3 was obtained by amplification with a primer pair F19/R110. p-Target was amplified with a primer pair F111/R111 to prepare a recombinant vector pTarget-tyrR. The knockout box UD3 and the recombinant vector pTarget-tyrR were electrically transformed into the HGB0-pCas. Bacterial colonies were subjected to PCR verification with a primer pair F112/R112, and the verified monoclone loses the pTarget-tyrR and the pCas9 to obtain an engineered strain HGC0.

The recombinant vector pAP-aroG^{fbr}-tyrA^{fbr}-ppsA-tktA was transformed into the E. coli HGC0 to obtain an engineered strain HGC.

The engineered strain HGC was inoculated into 50 mL of a seed culture medium and cultured at 220 rpm at 37° C. for 12 h to obtain a seed liquid. Then, the seed liquid was inoculated into a fermentation culture medium containing kanamycin with a final concentration of 50 μg/mL at an inoculation amount of 2% (v/v) and cultured at 220 rpm at 33° C. for 3 h, and the temperature was changed to 38° C. Synthesis of L-tyrosine was induced at 220 rpm at 38° C., fermentation was performed for 48 h, and 5.6 g/L tyrosine was accumulated in a shake flask.

All primer sequences are listed in Table 2.

TABLE 2
Primer sequences

| Primer | | |
|---|---|---|
| name | Primer sequence | |
| F11 | cgtctcgccaaactggaaaaatgg | SEQ ID NO: 13 |
| R11 | cttttcaccccgatttgggaggccttattg | SEQ ID NO: 14 |
| F12 | ctcccaaatcggggtgaaaaggtgccggatgatgtgaatcatcc | SEQ ID NO: 15 |
| R12 | caatggtttctggagcaaattcaggtctg | SEQ ID NO: 16 |
| F13 | atataccgaaagtacgtctggttttagagctagaaatagcaagttaaaataag | SEQ ID NO: 17 |
| | gctag | |
| R13 | cagacgtactttcggtatatactagtattatacctaggactgagctagctg | SEQ ID NO: 18 |
| F14 | ccagcaaacaaatggaaattactccgg | SEQ ID NO: 19 |
| R14 | gtctggtgtgatggacgtaaaccg | SEQ ID NO: 20 |
| F15 | ccaggcgttcaattaaggtttgcg | SEQ ID NO: 21 |
| R15 | gtttttatctcgccgaactgcgtcacgatcttgac | SEQ ID NO: 22 |
| F16 | gacgcagttcggcgagataaaaacagaaatcagggcag | SEQ ID NO: 23 |
| R16 | cgactctcgaactgctaacctgc | SEQ ID NO: 24 |
| F17 | attgccggaacacgcccacggttttagagctagaaatagcaagttaaaataag | SEQ ID NO: 25 |
| | g | |
| R17 | cgtgggcgtgttccggcaatactagtattatacctaggactgagctagctg | SEQ ID NO: 26 |
| F18 | ccaggagaaagcatcagcacc | SEQ ID NO: 27 |
| R18 | gcaatcagatacccagcccg | SEQ ID NO: 28 |
| F19 | cggaatcaacgttgatgattgcgg | SEQ ID NO: 29 |
| R19 | aggcatattcgcacttcggcgtaaagatatccg | SEQ ID NO: 30 |
| F110 | gccgaagtgcgaatatgcctgatggtgcaacacc | SEQ ID NO: 31 |
| R110 | gatctgtctgacgtcaccctcg | SEQ ID NO: 32 |
| F111 | ccacgggacagtacgcacatgttttagagctagaaatagcaagttaaaataag | SEQ ID NO: 33 |
| R111 | atgtgcgtactgtcccgtggactagtattatacctaggactgagctagctg | SEQ ID NO: 34 |
| F112 | gtccagccagttttagatgcccag | SEQ ID NO: 35 |
| R112 | gttacagtcgccaattccatccc | SEQ ID NO: 36 |
| F113 | gaagaaataaccggcgttcagcctgtgc | SEQ ID NO: 37 |
| R113 | cgttctgataattcatgatctttagctgtcttggtttgccc | SEQ ID NO: 38 |
| F114 | atgaattatcagaacgacgatttacgcatc | SEQ ID NO: 39 |
| R114 | gtgaggacatggtatatctccttttacccgcgacgcgcttttac | SEQ ID NO: 40 |
| F115 | gggtaaaaggagatataccatgtcctcacgtaaagagcttgcc | SEQ ID NO: 41 |
| R115 | gtaggtgagttacagcagttcttttgctttcgcaac | SEQ ID NO: 42 |
| F116 | gcaaaagaactgctgtaactcacctaccaaacaatgcccc | SEQ ID NO: 43 |
| R116 | gcaaccattggatcccaatgcttcgtttcg | SEQ ID NO: 44 |
| F117 | ggatccaatggttgctgaattgaccgcattac | SEQ ID NO: 45 |
| R117 | gttggacatggtatatctccttttactggcgattgtcattcgcc | SEQ ID NO: 46 |
| F118 | gccagtaaaaggagatataccatgtccaacaatggctcgtcac | SEQ ID NO: 47 |
| R118 | ggctgaacgccggttatttcttcagttcagccaggcttaacc | SEQ ID NO: 48 |

### Example 2: Exogenous Introduction of Fpk to Improve Synthesis of L-Tyrosine

In order to improve the utilization of glucose, fpk from Bifidobacterium adolescentis was heterologously expressed to directionally guide glucose to a shikimic acid pathway so as to increase precursor supply of the shikimic acid pathway. In order to prevent too long plasmids from affecting gene expression efficiency, genes ppsA and tktA and a strong promoter PJ₂₃₁₁₁₉ were linked for integration on an E. coli genome.

(1) Preparation of an Engineered Strain HGD0

With the engineered strain HGC0 constructed in Example 1 as an original strain, electrically transformed competent cells HGC0-pCas of HGC0 containing a plasmid pCas were constructed by the same method. A gene tktA was integrated by the same gene knockout method. With an E. coli K12 genome as a template, the gene tktA was amplified with a primer pair F23/R23, and upstream and downstream homologous arms of dadx-cvra were amplified with primer pairs F24/R24 and F25/R25, respectively. With the gene tktA and the upstream and downstream homologous arms of dadx-cvra as templates, a knock-in box UTD was obtained by amplification of the tktA and the upstream and downstream homologous arms with a primer pair F24/R25. p-Target was amplified with a primer pair F26/R26 to prepare a recombinant vector pTarget-dadx-cvra. The knock-in box UTD and the recombinant vector pTarget-dadx-cvra were electrically transformed into the HGC0-pCas. Bacterial colonies were subjected to PCR verification with a primer pair F27/R27, and the verified monoclone loses the pTarget-dadx-cvra and the pCas9 to obtain an engineered strain HGD0.

(2) Preparation of an Overexpression Plasmid pAP-aroG^{fbr}-tyrA^{fbr}-Fpk and Engineered Strains HGE0 and HGE

Electrically transformed competent cells HGD0-pCas of HGD0 containing a plasmid pCas were constructed by the same method. A gene ppsA was integrated by the same method. With an E. coli K12 genome as a template, the gene ppsA was amplified with a primer pair F28/R28, upstream and downstream homologous arms of ykgh-betA were amplified with primer pairs F29/R29 and F210/R210, respectively, and a knock-in box UPD was obtained by amplification of the ppsA and the upstream and downstream homologous arms with a primer pair F29/R210. p-Target was amplified with a primer pair F211/R211 to prepare a recombinant vector pTarget-ykgh-betA. The knock-in box UPD and the recombinant vector pTarget-ykgh-betA were electrically transformed into the HGD0-pCas. Bacterial colonies were subjected to PCR verification with a primer pair F212/R212, and the verified monoclone loses the pTarget-ykgh-betA and the pCas9 to obtain an engineered strain HGE0.

With synthetic fpk as a template, amplification was performed with a primer pair F21/R21, followed by purification and recovery. With the recombinant vector pAP-aroG^{fbr}-tyrA^{fbr} constructed in Example 1 as a template, amplification was performed with a primer pair F22/R22, and fragments were recovered. A fragment fpk and the vector pAP-aroG^{fbr}-tyrA^{fbr} skeleton were reconstructed by a Gibson assembly method to obtain a recombinant vector, the recombinant vector was transformed into E. coli JM109, and a plasmid was extracted and sequenced for verification to obtain a correct recombinant vector pAP-aroG^{fbr}-tyrA^{fbr}-fpk.

The pAP-aroG^{fbr}-tyrA^{fbr}-fpk was transformed into the E. coli HGE0 to obtain an engineered strain HGE.

The engineered strain HGE was inoculated into 50 mL of a seed culture medium and cultured at 220 rpm at 37° C. for 12 h to obtain a seed liquid. Then, the seed liquid was inoculated into a fermentation culture medium containing kanamycin with a final concentration of 50 μg/mL at an inoculation amount of 2% (v/v), and cultured at 220 rpm at 33° C. for 3 h, and the temperature was changed to 38° C. Synthesis of L-tyrosine was induced at 220 rpm at 38° C., fermentation was performed for 48 h, and 6.0 g/L L-tyrosine was accumulated in a shake flask.

All primer sequences are listed in Table 3.

TABLE 3
Primer sequences

| Primer | | |
|---|---|---|
| name | Primer sequence | |
| F21 | ggtaaaaggagatataccatgactaaccctgtaatcggtactcc | SEQ ID NO: 49 |
| R21 | gtttggtaggtgagttattcattgtcaccagcagtagcagc | SEQ ID NO: 50 |
| F22 | ggtgacaatgaataactcacctaccaaacaatgcccc | SEQ ID NO: 51 |
| R22 | cagggttagtcatggtatatctccttttacccgcgacgcgcttttactgcattc | SEQ ID NO: 52 |
| F23 | ttgacagctagctcagtcctaggtataatactagtaaagaggagaaaaagct | SEQ ID NO: 53 |
| | tatgtcctcacgtaaagagcttgc | |
| R23 | ttacagcagttcttttgctttcgcaac | SEQ ID NO: 54 |
| F24 | gtgatatcgccaataccggattacg | SEQ ID NO: 55 |
| R24 | ggactgagctagctgtcaatgcggtgagttcaggttccgg | SEQ ID NO: 56 |
| F25 | gcaaaagaactgctgtaacaggcgttctacataaaacgcttacgc | SEQ ID NO: 57 |
| R25 | ggcgatgtgttgtgtgtaattgg | SEQ ID NO: 58 |
| F26 | ggcgaagaatatcatccatggttttagagctagaaatagcaagttaaaataa | SEQ ID NO: 59 |
| | gg | |
| R26 | catggatgatattcttcgccactagtattatacctaggactgagctagctg | SEQ ID NO: 60 |
| F27 | cgcattttgactgggttcggc | SEQ ID NO: 61 |
| R27 | gcggcactgtttcgtgataacc | SEQ ID NO: 62 |
| F28 | ttgacagctagctcagtcctaggtataatactagtaaagaggagaaaaagct | SEQ ID NO: 63 |
| | tatgtccaacaatggctcgtcac | |
| R28 | gattgagagttttatttcttcagttcagccaggcttaac | SEQ ID NO: 64 |
| F29 | gtatctcatcgagaacttgcctgcc | SEQ ID NO: 65 |
| R29 | gactgagctagctgtcaaaccgttccagagagggggacc | SEQ ID NO: 66 |
| F210 | gaagaaataaaactctcaatctgatcggttcctgc | SEQ ID NO: 67 |
| R210 | gtgcggattaaatcccgcgac | SEQ ID NO: 68 |
| F211 | ggtgaaaacgactatcacgggttttagagctagaaatagcaagttaaaataa | SEQ ID NO: 69 |
| | gg | |
| R211 | ccgtgatagtcgttttcaccactagtattatacctaggactgagctagctg | SEQ ID NO: 70 |
| F212 | cggatacaatgaccagttcctgg | SEQ ID NO: 71 |
| R212 | Cggtttccagtgccacgtc | SEQ ID NO: 72 |

### Example 3: Modification of Acetic Acid Pathway to Improve Utilization of Glucose

When the HGE strain constructed in Example 2 was fermented in a shake flask, it was found that the content of acetic acid accumulated in the shake flask was 1.2 g/L within 48 h, resulting in serious waste of carbon resources. Therefore, an acetic acid pathway of E. coli was modified. A gene poxB encoding pyruvate oxidase (PoxB) in E. coli was knocked out.

With an E. coli K12 genome as a template, an upstream homologous arm U1 and a downstream homologous arm D1 of the gene poxB were amplified with primer pairs F31/R31 and F32/R32, respectively, and fragments were purified. With purified fragments U1 and D1 as templates, a knockout box UD1 was obtained by amplification with a primer pair F31/R32, and fragments were purified. In order to obtain pTarget-poxB for knocking out the poxB, amplification was performed with a primer pair F33/R33 with p-Target stored in a laboratory as a template, and fragments were purified. A purified fragment was transformed into E. coli JM109, and a plasmid was extracted and sequenced for verification to obtain a correct recombinant vector pTarget-poxB.

With the engineered strain HGE as an original strain, electrically transformed competent cells HGE-pCas of HGE containing a plasmid pCas were constructed by the same method. A gene poxB of E. coli HGE was knocked out by the same experimental method in Example 1. Bacterial colonies were subjected to PCR verification with a primer pair F34/R34, and the verified monoclone loses the pTarget-poxB and the pCas9 to obtain an engineered strain HGF0.

The recombinant vector pAP-aroG^{fbr}-tyrA^{fbr}-fpk in Example 2 was transformed into the E. coli HGF0 to obtain an engineered strain HGF.

The engineered strain HGF was inoculated into 50 mL of a seed culture medium and cultured at 220 rpm at 37° C. for 12 h to obtain a seed liquid. Then, the seed liquid was inoculated into a fermentation culture medium containing kanamycin with a final concentration of 50 μg/mL at an inoculation amount of 2% (v/v), and cultured at 220 rpm at 33° C. for 3 h, and the temperature was changed to 38° C. Synthesis of L-tyrosine was induced at 220 rpm at 38° C., fermentation was performed for 48 h, and 6.2 g/L L-tyrosine was accumulated in a shake flask, where the accumulation content of acetic acid was only 0.45 g/L, which was effectively reduced by 62.5%.

All primer sequences are listed in Table 4.

TABLE 4
Primer sequences

| Primer name | Primer sequence | |
|---|---|---|
| F31 | ggcaacactttgccgttgtgg | SEQ ID NO: 73 |
| R31 | cataatcgccgaactggcgaaaacaaactggc | SEQ ID NO: 74 |
| F32 | tcgccagttcggcgattatgcgagaaccaaatcc | SEQ ID NO: 75 |
| R32 | cgatgagtggcgtaactatccgg | SEQ ID NO: 76 |
| F33 | ggtgaaaatagcgtcatcgggttttagagctagaaatagcaagttaaaat | SEQ ID NO: 77 |
| | aagg | |
| R33 | ccgatgacgctattttcaccactagtattatacctaggactgagctagctg | SEQ ID NO: 78 |
| F34 | gtcaacatgcagcgccagatt | SEQ ID NO: 79 |
| R34 | gatctacaacgtgcgtacgcc | SEQ ID NO: 80 |

### Example 4: Modification of Aromatic Amino Acid Transport System

(1) Preparation of Engineered Strains HGG0 and HGG

Through determination of the intracellular content of tyrosine in the strain HGF in Example 3, it was found that the intracellular concentration of tyrosine in the HGF was 972.7% higher than that of wild-type E. coli K12 as a control group. Therefore, an aromatic amino acid transport system of the HGF was modified. A gene aroP encoding permease of an aromatic amino acid transporter AroP in E. coli was knocked out. With an E. coli K12 genome as a template, an upstream homologous arm U1 and a downstream homologous arm D1 of the gene aroP were amplified with primer pairs F41/R41 and F42/R42, respectively, and fragments were purified. With purified fragments U1 and D1 as templates, a knockout box UD1 was obtained by amplification with a primer pair F41/R42, and fragments were purified. In order to obtain pTarget-aroP for knocking out the aroP, amplification was performed with a primer pair F43/R43 with p-Target stored in a laboratory as a template, and fragments were purified. A purified fragment was transformed into E. coli JM109, and a plasmid was extracted and sequenced for verification to obtain a correct recombinant vector pTarget-aroP.

With the engineered strain HGF0 as an original strain, electrically transformed competent cells HGF0-pCas of HGF0 containing a plasmid pCas were constructed by the same method. A gene aroP of E. coli HGF0 was knocked out by the same experimental method in Example 1. Bacterial colonies were subjected to PCR verification with a primer pair F44/R44, and the verified monoclone loses the pTarget-aroP and the pCas9 to obtain an engineered strain of E. coli HGG0.

The recombinant vector pAP-aroG^{fbr}-tyrA^{fbr}-fpk in Example 2 was transformed into the E. coli HGG0 to obtain an engineered strain HGG.

(2) Preparation of Overexpression Plasmid pAP-aroG^{fbr}-yddG-tyrA^{fbr}-Fpk and Engineered Strains HGH0 and HGH

A gene tyrP was knocked out by the same method to block the synthesis of a tyrosine specific transport protein. With the engineered strain HGG0 as an original strain, electrically transformed competent cells HGG0-pCas of HGG0 containing a plasmid pCas were constructed by the same method. A gene tyrP was knocked out by the same method to block the synthesis of tryptophan. With an E. coli K12 genome as a template, upstream and downstream homologous arms of the tyrP were amplified with primer pairs F45/R45 and F46/R46, respectively, and a knockout box UD1 was obtained by amplification with a primer pair F45/R46. p-Target was amplified with a primer pair F47/R47 to prepare a recombinant vector pTarget-tyrP. The knockout box UD1 and the recombinant vector pTarget-tyrP were electrically transformed into the HGG0-pCas. Bacterial colonies were subjected to PCR verification with a primer pair F48/R48, and the verified monoclone loses the pTarget-tyrP and the pCas9 to obtain an engineered strain HGH0.

With an E. coli K12 genome as a template, a fragment yddG was amplified with a primer pair F49/R49. With the vector pAP-aroG^{fbr}-tyrA^{fbr}-fpk as a template, amplification was performed with a primer pair FP410/RP410, and a product was purified. A fragment yddG and the vector pAP-aroG^{fbr}-tyrA^{fbr}-fpk skeleton were reconstructed by a Gibson assembly method to obtain a recombinant vector, the recombinant vector was transformed into E. coli JM109, and a plasmid was extracted and sequenced for verification to obtain a correct recombinant vector pAP-aroG^{fbr}-yddG-tyrA^{fbr}-fpk. The recombinant vector pAP-aroG^{fbr}-yddG-tyrA^{fbr}-fpk was transformed into the E. coli HGH0 to obtain an engineered strain HGH.

The engineered strain HGH was inoculated into 50 mL of a seed culture medium and cultured at 220 rpm at 37° C. for 12 h to obtain a seed liquid. Then, the seed liquid was inoculated into a fermentation culture medium containing kanamycin with a final concentration of 50 μg/mL at an inoculation amount of 2% (v/v), and cultured at 220 rpm at 33° C. for 3 h, and the temperature was changed to 38° C. Synthesis of L-tyrosine was induced at 220 rpm at 38° C., fermentation was performed for 48 h, and 6.9 g/L tyrosine was accumulated in a shake flask.

All primer sequences are listed in Table 5.

TABLE 5
Primer sequences

| Primer | | |
|---|---|---|
| name | Primer sequence | |
| F41 | cgtgagtatttgcgtgagctgc | SEQ ID NO: 81 |
| R41 | acgaggtttctctctctacgccctcacccg | SEQ ID NO: 82 |
| F42 | cgtagagagagaaacctcgtgcggtggttg | SEQ ID NO: 83 |
| R42 | ggtcttaccaatttcatgtctgtgacg | SEQ ID NO: 84 |
| F43 | aatcaccacaaagaatacgggttttagagctagaaatagcaagttaaaat | SEQ ID NO: 85 |
| | aagg | |
| R43 | cagctagctcagtcctaggtataatactagtaatcaccacaaagaatacgg | SEQ ID NO: 86 |
| F44 | ttggcgcaggtaaagttcgt | SEQ ID NO: 87 |
| R44 | tgttttgccagttcgcgttc | SEQ ID NO: 88 |
| F45 | gcggcgaaggtctgtattttatcga | SEQ ID NO: 89 |
| R45 | ctatctgagctttcttctgtcctgacgatctttatgag | SEQ ID NO: 90 |
| F46 | cgtcaggacagaagaaagctcagatagcctcaaattccttattgggtgc | SEQ ID NO: 91 |
| R46 | ctggcttttcaacatatggccgatac | SEQ ID NO: 92 |
| F47 | agaaaatatatgccaccagggttttagagctagaaatagcaagttaaaat | SEQ ID NO: 93 |
| | aagg | |
| R47 | cagctagctcagtcctaggtataatactagtagaaaatatatgccaccagg | SEQ ID NO: 94 |
| F48 | gagcagcatgaagaagagaaactgttc | SEQ ID NO: 95 |
| R48 | ggcgtcagagaaagagatgacgc | SEQ ID NO: 96 |
| F49 | ggtaaaaggagatataccatgacacgacaaaaagcaacgc | SEQ ID NO: 97 |
| R49 | gtttggtaggtgagttaaccacgacgtgtcgccag | SEQ ID NO: 98 |
| F410 | cgtcgtggttaactcacctaccaaacaatgcccc | SEQ ID NO: 99 |
| R410 | ctttttgtcgtgtcatggtatatctccttttacccgcgacgcgcttttac | SEQ ID NO: 100 |

### Example 5: Optimization of Fermentation in 5-L Fermentation Tank

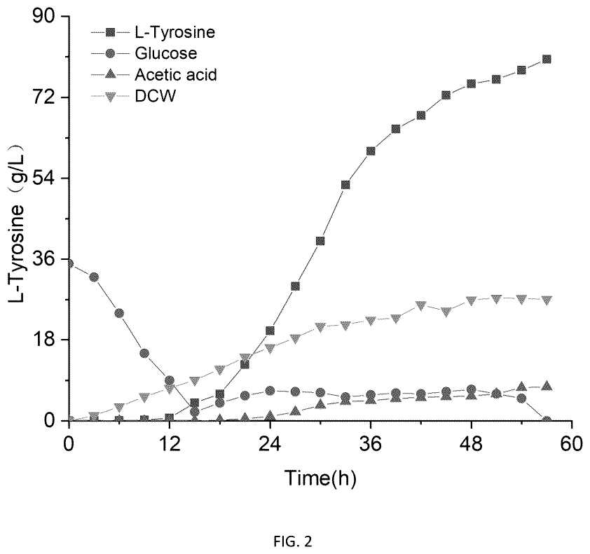

The engineered strain HGH constructed in Example 4 was inoculated into 50 mL of a seed culture medium and cultured at 220 rpm at 37° C. for 12 h to obtain a primary seed liquid, the primary seed liquid was inoculated into 50 mL of a secondary seed liquid at an inoculation amount of 2% (v/v), and then, the secondary seed liquid was inoculated into 2.5 L of a fermentation culture medium containing kanamycin with a final concentration of 50 μg/mL at an inoculation amount of 2% (v/v). With the initial rotation speed controlled at 300 rpm, the seed liquid was cultured at 33° C. for 12 h until the OD₆₀₀ value was 20-23, heated to 38° C. and continuously cultured for 48-55 h to obtain a fermentation liquid. In a whole fermentation process, the pH value was controlled at 6.4-6.6 by fed-batch 50% ammonia water; when the dissolved oxygen (DO) value was decreased to 20%, the rotation speed or the ventilation capacity was gradually increased to maintain the DO value at 20% or above; and when the glucose in a culture medium was depleted, a feeding procedure was started, and 750 g/L glucose was added to perform fed-batch fermentation, where the concentration of glucose was maintained at about 5-8 g/L. After completion of fermentation, the content of tyrosine was determined. Finally, as shown in FIG. 2, when the engineered strain HGH was subjected to fed-batch fermentation in a 5 L fermentation tank for 55 h, the accumulation content of tyrosine was 80.5 g/L, and the production intensity was 1.46 g/L/h.

Although the present disclosure has been disclosed as above through the preferred examples, the examples are not intended to limit the present disclosure. For any person familiar with the art, various changes and modifications can be made without departing from the spirit and scope of the present disclosure. Therefore, the protection scope of the present disclosure should be as defined in the claims.

What is claimed is:

1. Recombinant Escherichia coli for efficient synthesis of L-tyrosine, wherein the recombinant E. coli is obtained by using E. coli as an original strain and knocking out a gene pheA encoding and fusing chorismate mutase/prephenate dehydratase, a gene trpE encoding an anthranilate synthetase subunit TrpE, a gene tyrR encoding a DNA binding transcription double regulator TyrR, a gene poxB encoding pyruvate oxidase, a gene aroP encoding permease of an aromatic amino acid transporter AroP, a gene tyrP encoding a tyrosine and H (+) sympoter, a gene aroG^{fbr} freely expressing and encoding 3-deoxy-D-arabino-heptulosonate-7-phosphate synthase, a gene tyrA^{fbr} encoding chorismate mutase and prephenate dehydrogenase, a gene fpk encoding phosphoketolase and a gene yddG encoding an amino acid exportin YddG on an E. coli genome;
and the gene pheA has a nucleotide sequence set forth in SEQ ID NO:1, the gene trpE has a nucleotide sequence set forth in SEQ ID NO:2, the gene aroG^{fbr} has a nucleotide sequence set forth in SEQ ID NO:3, the gene tyrA^{fbr} has a nucleotide sequence set forth in SEQ ID NO:4, the gene tyrR has a nucleotide sequence set forth in SEQ ID NO:5, the gene fpk has a nucleotide sequence set forth in SEQ ID NO:8, the gene poxB has a nucleotide sequence set forth in SEQ ID NO:9, the gene aroP has a nucleotide sequence set forth in SEQ ID NO:10, the gene tyrP has a nucleotide sequence set forth in SEQ ID NO:11, and the gene yddG has a nucleotide sequence set forth in SEQ ID NO:12.

2. The recombinant E. coli according to claim 1, wherein the recombinant E. coli integrates a gene ppsA expressing phosphoenolpyruvate synthetase and a gene tktA encoding transketolase 1; and the gene ppsA has a nucleotide sequence set forth in SEQ ID NO:6, and the gene tktA has a nucleotide sequence set forth in SEQ ID NO:7.

3. The recombinant E. coli according to claim 2, wherein the gene ppsA is integrated at a site ykgh-betA and the gene tktA is integrated at a site dadx-cvra on the E. coli genome.

4. The recombinant E. coli according to claim 3, wherein the gene ppsA and the gene tktA are initially expressed by a promoter PJ₂₃₁₁₁₉.

5. The recombinant E. coli according to claim 1, wherein a heat induced expression vector pAP-B03 is used as an expression plasmid.

6. The recombinant E. coli according to claim 1, wherein E. coli K12, E. coli BL21, E. coli DH5α, E. coli JM109 or E. coli WSH-Z06 is used as the original strain.

7. A method for producing L-tyrosine, comprising using the recombinant E. coli according to claim 1 to produce the L-tyrosine by fermentation.

8. The method according to claim 7, comprising inoculating the recombinant E. coli into a fermentation system, culturing at 32-34° C. for 3-12 hours and fermenting at 200-220 rpm at 36-40° C. for 48-60 hours.

9. The method according to claim 7, wherein a fermentation system comprises 30-40 g/L glucose, 3-7 g/L (NH₄)₂SO₄, 1-5 g/L KH₂PO₄, 1-5 g/L MgSO₄·7H₂O, 1-2 g/L sodium citrate, 0.5-1.5 g/L NaCl, 0.05-0.1 g/L vitamin B₁, 0.1-0.12 g/L FeSO₄·7H₂O, 1-3 g/L yeast powder, 2-6 g/L peptone and 1-2 mL/L trace element nutrient solution.


SEQUENCE LISTING (101 sequences)

SEQ ID NO: 1 - DNA, 1161 bp, Escherichia coli
  source 1..1161: mol_type = other DNA
atgacatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccaccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcaaatcagcgacaaaacttcacccgatttgtggtgttggcgcgtaaagccattaacgtgtctgatcaggttccggcgaaaaccacgttgttaatggcgaccgggcaacaagccggtgcgctggttgaagcgttgctggtactgcgcaaccacaatctgattatgacccgtctggaatcacgcccgattcacggtaatccatgggaagagatgttctatctggatattcaggccaatcttgaatcagcggaaatgcaaaaagcattgaaagagttaggggaaatcacccgttcaatgaaggtattgggctgttacccaagtgagaacgtagtgcctgttgatccaacctga

SEQ ID NO: 2 - DNA, 1563 bp, Escherichia coli
  source 1..1563: mol_type = other DNA
tcagaaagtctcctgtgcatgatgcgcggtggcaatagcgcgcagtacagcgcgggctttgttacgggtttcgtcggcttccgactgcggaacagaatcaaggactacaccagcacccgcttgcacggtggcgataccgttttccaccagcgccgagcggatcacaatgcaggtgtcgagatcgccatgcgcggtgaaataacctaccgcgccgccgtagctgccgcggcgacgaccttccgcctcggcaattaactgcatagcgcgtactttcggcgcaccgcttaacgtccccatattcatacaggcgcgataagcgtgcagggcgtcaagatcgtgacgcagttcgccgactacgcgagagacgaggtgcatcacataggaataacggtcaactttggtgagatcggcgacgtagcggctgccgggggtgcaaatgcgtgccagatcattacgggcgagatcaaccagcatcagatgttcagacagctctttatgatcggtacgcatttccagttcaatacggctgtcgagatctctgtccagtgaaccatcggcgcgacgaccgcgtgggcgtgttccggcaatcgggtagatctcaatctggcggctggtggcatcatacttgagcgagctttccggcgacgcgccaaatagggtgaaatcattatcctgcataaaaaacatgtacgggctgggattactctttttcagcacgtaataggccgccagcggtgacgggcagggcagagagaaacggcgagatggcaccacctggaaaatttctccagcgcgaatcgctttttgcaacaaacgcactacgccaccgaactcttcatcgctctgattacattcacaacgcatatgcggcacggaaaccactggcagcggcggcgcggcttcggtcagttgctgacgtagttcgttcaggcgagcagtgagacgttgtttttcttcttcattcggagcaaacaggctggcctgaatacgggtgctttttttctgatggtcaatcaccatcagcgtttcagcgagataaaaacagaaatcagggcagttattttccgctgacagttgcggtaaatcttcaaatcccgccacaaggtcataagagaacaggccgccgaagaacatggcttctcgttcttccttcggtacattcaacagattctgcaataaacggaaagcgtcaaaaaccgaaagggagcataagcgggcgtcttcatccagcagtggactgacaggggggaagcgcagcacacggcagtttggtgattgttcactttccacacccgcaggcagggcgttatccagtagtgccaggagggcttcgccgttgccggaaagtgcctggattgtgacagtgtcacctaaagctgtaatgcgcagcgcactgtctaccagcagcaggctttttaaatcatctttgctgtcgatatctgcggattccagcagcagcgttgccggacgatccccacacaactggtgaaaaagcgcggtgggattgtcgcgataagcgccttcgcaggttagcagttcgagagtcggtttttgtgtttgcat

SEQ ID NO: 3 - DNA, 1053 bp, Escherichia coli
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 4 - DNA, 1122 bp, Escherichia coli
  source 1..1122: mol_type = other DNA
ttactggcgattgtcattcgcctgacgcaataacacgcggctttcactctgaaaacgctgtgcgtaatcgccgaaccagtgctccaccttgcggaaactgtcaataaacgcctgcttatcgccctgctccagcaactcaatcgcctcgccgaaacgcttatagtaacgtttgattaacgccagattacgctctgacgacataatgatgtcggcataaagctgcggatcctgagcaaacagtcgcccgaccatcgccagctcaaggcggtaaatcggcgaagagagcgccagaagttgctcaagctgaacattttcttctgccaggtgcagcccgtaagcaaaagtagcaaagtggcgcagtgcctgaataaacgccatattctgatcgtgctcgacggcgctaatacgatgcagccgagcgccccagacctgaatttgctccagaaaccattggtatgcttccggtttacgtccatcacaccagaccacaacttgctttgccaggctaccgctgtccggaccgaacatcgggtgtagccccagcaccggaccatcatgcgccaccagcatggcctgtaatggcccatttttcactgatgccagatcgaccagaatacaatctttcggtaaaggcggtaatttgccaataacttgctcagtaacgtggattggcacactaacaatcaccattccggcatcggcaacaatatcagccgctcgatcccagtcatgttgctccagaatccgcacctgataacccgagagggtcagcatcttctcgaacaggcgtcccatctgaccgccaccgccgacgataaccaccggacgcagtgacggacaaagtgttttaaatcctttgtcgttttcactggagtaagattcacgcatcacccgacgcaaaacatcctcaatcagatctggcggtacacccagagcttccgcctctgcacgacgcgaggccaacatagatgcctcgcgctccggaacataaataggcagtccaaagcggcttttcacctcgcccacttcagcaaccagttccagacgcttcgctaataaattcagcagcgctttatcgacttcatcaatttgatcgcgtaatgcggtcaattcagcaaccat

SEQ ID NO: 5 - DNA, 1542 bp, Escherichia coli
  source 1..1542: mol_type = other DNA
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 6 - DNA, 2379 bp, Escherichia coli
  source 1..2379: mol_type = other DNA
ttatttcttcagttcagccaggcttaaccaggtttgcaccacggtgtccgggttcagagacaggctatcgatcccctcttccatcaaccatgcggcaaagtcttcgtggtcggacggaccctgaccgcaaatcccgacatatttgccctgtttcttcgcggcacggatagccatcgacagcagtgctttcaccgcatcgttgcgctcatcgaacaattcagacaccacgccggagtcacggtccagaccgagcgccagctgcgtcatatcgtttgagccaattgagaagccgtcgaaatattcgaggaactgctcggccagcaaggcgttggacgggatttcacacatcatgatgattttcagcccgttctcgccacgtttcagcccctgacgcgccagttcttcaaccaccgctttcgcctgatctacggtacgcacgaacgggatcatgatctcaacgttggtcagtcccatgtcgttgcgcacacgtttcactgcttcacactccagcgcgaaacagtcgcggaagctgtcggaaacatagcggcccgcgccacggaagccgagcatcgggttctcttcatctggctcgtaacgctcaccaccgaccaggttggcatattcgttcgatttaaaatcagagagacggacaatgacgcgcttcggataaaacgcggcacccagcgtcgcgatcccttcagtcagacgaccaacgtaaaattcacgcggagaatcaaaacctttcatcatctcgcggatttcgttttgcaactgcggttcctgatcgtcaaactcaagcagtgcgcgtgggtggacgccaatcatacggttgatgataaattccagacgcgcaaggcccacgccttcgttcggtaggcaggcgaagtcgaaagcacggtccgggttaccgacgttcatcatcactttcaacggcagatccggcatcgtttctacgctggagcttttcacgctaaattccagcaactccgcatagacgtaaccggtatcaccttcggcacaagaaacagtgacgttctcaccgtctttcatccgttctgttgcatctccacagcccactaccgccggaatgcccagttcacgagcgatgatcgccgcgtgacaggtacgaccgccacggttggtgacgatggcagatgctttcttcatgatcggttcccagtccgggtcggtcatgtcagtaaccagcacgtcgccaggttcgatgcggttcatttcgctgatgtcatggatgactttcaccggacccgcaccgatgcgatgaccgatagcacggccttcggcgataatcttaccctgtgaatgcagcgtataacgctccatgacctgaccgcgtgagcgcacggtttccggacgcgcctgcacaatgaacagtttaccggtgtggccatctttcgcccactcaatatccatcgggcgaccgtagtgtttctcaatttgtacggcctgttttgccagttcctgcacttcttcgttggtcagcgagaagatgtcacgctgttcctgcggtacgtcttcgattttaacctgcttgccgtgctcctgggtcggcgcgtaaaccatgcggatttttttcgaccccatggtgcggcgcacgatagccgggcgattcgccgccagtgtcggtttatgcacgtaaaactcatccgggttaaccgcaccctgcacgaccatctcaccaaggccccatgcggaagtgataaacaccacctggtcaaagccggattcggtatcaatggagaacatcacgccagatgatgcgaggtcagagcgcaccatccgttgaacaccggcggagagcgccacaccacggtgatcgtaaccctggtgcacacgataagagatggcgcgatcgttaaacagagaagcaaatacatgtttcactgccacgagaacggcgtcaaaaccctgaacgttgaggaaggtttcctgctgaccggcaaaagaagcgtccggcatatcttctgcggtggcggaggagcgcaccgcaaaagaggcgttttcgtcatcggcggaaagctgtgcataggcttcgcggatggcgttttccagctcaggctggaagggagtgtcgataatccactggcggatttgcgcgcccgctttcgcaagctgagtaacatcgtcaatatccgttttatccagcagttcataaatgcgctggtttacgccgctttggtccagaaactggttaaacgcgtcggcggttgtggcgaaaccattcggaacggaaacacccattccggaaagattagtaatcatttcacccagggaggcatttttgcccccaaccctgtctacatcattcatgccgagttggttataccaaagcaccagcggtgacgagccattgttggacat

SEQ ID NO: 7 - DNA, 1992 bp, Escherichia coli
  source 1..1992: mol_type = other DNA
ttacagcagttcttttgctttcgcaacaacgttatcaacagtgaagccgaactcttcaaacagcagctctgccggagcagattcaccgaaggtggtcataccgacgatagcaccgttcaggccaacatacttgtaccagtagtcagcaatacccgcttctacagcaacgcgtgcagtaaccgctttcggcagtacggattcacggtaagcagcatcctgcttgtcaaatgcgtcggtagacggcatggacaccacgcgcgctttcacgccttcggcagtcagtttttcgtaggcagcaacagccagttcaacttctgaaccggtagcgatgaaaatcagttccggctgaccggcgcagtctttcagcacataaccaccgcgcgcgatgtttgccagttgctcttcagttcgttcctgctgcgccaggttctgacgggagaggatcagtgcggtcgggccgtcctgacgctcaacaccgtatttccacgcgaccgcggattcaacctggtcacacggacgccatgtagacatgttcggggttacgcgcagagaagcgacctgctcaaccggctggtgagtcgggccgtcttcgcccagaccgatggagtcgtgggtgtaaaccatcacctgacgctgtttcatcagcgcagccatacgtacggcgttacgtgcgtattccacgaacatcaggaaggtggaggtgtacggcaggaagccaccgtgcagggagataccgttagcaatcgcggtcataccgaactcgcgaacaccgtagtggatgtagttacccgcagcatcttcgttgattgctttagaaccagaccacagggtcaggttagacggcgccaggtcagcagaaccgccgaggaattccggcaacagcggaccgaacgcttcgatagcattctgagacgctttacggctggcgattttcgccggattagcctgcagtttagcgatgaactctttcgctttagcgtcgaagtcagacggcatttcgcctttcatacggcgggtaaattcagcggcttcctgcggataagctttcgcgtaagcagcgaatttctcgttccatgcggattctttcgcctggcctgcttctttcgcatcccactgagcatagatttcagacgggatttcgaacggcgcatatttccagcccagttgttcgcgggtcagggcaatttcagcgtcgcccagcggcgcaccgtgggagtcgtgggtaccggctttgttcggggaaccgaaaccgatgatggttttgcacatcagcagggaaggtttgtcagtcactgcgcgcgcttcttctactgcgcgtttgatagatgccgcgtcatgaccgtcgatgtcgcgaataacgtgccagccgtaagcttcgaaacgcattgcggtgtcgtcggtgaaccagccttcaacgtgaccatcgatagaaataccgttgtcatcgtagaatgcaatcagtttacccagcttcagcgtacccgccagagagcaaacttcgtgggagatgccttccatcatgcagccgtcgcccatgaaggcgtaggtgtagtggtcgacaatgtcgtggcccggacggttaaactgcgccgccagcgttttttctgcaatcgccataccgactgcgttggcaataccctgacccagcggaccggtggtggtttccacaccagcggtgtaacccacttccgggtgacccggagttttagagtgcagctgacggaagtttttcagttcttccatcggcagatcgtaaccggtgaggtgcagcaggctgtagatcagcatggagccgtggccgttggacagcacgaagcggtcacggtcagcccaggacggattctgcgggttgtgtttcaggaaatcacgccacaggacttcggcaatgtcagccatacccataggggcacccgggtgaccggatttggctttctgtactgcgtccatgctcagcgcacgaatagcattggcaagctctttacgtgaggacat

SEQ ID NO: 8 - DNA, 2478 bp, Bifidobacterium adolescentis
  source 1..2478: mol_type = other DNA
atgactaaccctgtaatcggtactccttggcaaaagttggatagaccagtttcagaagaagcaatcgaaggtatggataaatattggagagttaccaactatatgtccataggtcaaatctacttgagaagtaacccattgatgaaggaaccttttactagagatgacgttaagcatagattagtcggtcactggggtactacaccaggtttgaacttcttgttggcccatatcaacagattgatcgctgatcaccaacaaaacaccgtttttataatgggtccaggtcatggtggtccagctggtacttcccaaagttatgttgacggtacttacactgaatactacccaaacataacaaaagatgaagctggtttgcaaaagtttttcagacaattctcctatccaggtggtatccctagtcattttgcaccagaaacccctggttcaattcacgaaggtggtgaattgggttatgctttatctcatgcttacggtgcagtaatgaataacccatcattgtttgttccttgtattataggtgacggtgaagccgaaacaggtccattagctaccggttggcaatctaacaaattggtcaatccaagaactgatggtatcgtattgcctatcttgcatttgaacggttacaagattgcaaatccaacaatcttggccagaatatctgatgaagaattacatgactttttccgtggtatgggttatcacccttacgaatttgttgccggtttcgataatgaagaccacatgtctatccacagaagattcgctgaattgttcgaaactatcttcgatgaaatttgtgacataaaagctgctgctcaaaccgatgacatgactagaccattctaccctatgttgatttttagaactccaaagggttggacatgccctaagttcatcgatggtaaaaagacagaaggttcctggagagcacatcaagttccattagctagtgcaagagataccgaagaacactttgaagtcttgaaaggttggatggaatcttacaagcctgaagaattattcaatgcagatggttcaattaaagatgacgttacagcctttatgccaaagggtgaattgagaataggtgccaatcctaacgctaatggtggtgttatcagagaagatttgaaattgccagaattagaccaatatgaagtaactggtgttaaggaatacggtcatggttggggtcaagttgaagcccctagagctttgggtgcatattgtagagatatcattaaaaataacccagactcctttagaatattcggtcctgatgaaacagctagtaacagattgaacgcaacttatgaagtaaccgataagcaatgggacaatggttacttgtctggtttagttgatgaacacatggcagtcactggtcaagtaacagaacaattatcagaacaccaatgcgaaggtttcttggaagcatatttgttaacaggtagacatggtatttggtcttcatacgaatcttttgtacatgttatcgattcaatgttgaaccaacacgccaaatggttagaagctactgttagagaaataccttggagaaagcctatctccagtgttaacttgttagtctcttcacatgtatggagacaagatcataatggtttttctcaccaagacccaggtgtcacatcattgttgattaataagaccttcaataacgatcacgttaccaacatctattttgccactgacgctaacatgttgttggctatctctgaaaagtgcttcaagtcaactaacaaaatcaatgcaatattcgccggtaaacaaccagcacctacatgggttaccttggatgaagccagagctgaattagaagctggtgctgctgaatggaaatgggcttctaatgcagaaaataacgatgaagttcaagttgtcttggcatccgccggtgacgtcccaacacaagaattgatggccgctagtgatgctttgaacaaaatgggtattaagtttaaagtagttaacgtcgtagatttgttgaagttacaatcaagagaaaacaacgatgaagcattgactgacgaagagtttactgaattgtttactgctgataaaccagtattgtttgcatatcattcctacgcccaagatgttagaggtttgatctatgatagaccaaaccatgacaatttccacgttgtcggttacaaagaacaaggttcaaccactacaccttttgatatggtcagagtaaatgatatggacagatatgcattgcaagcagccgctttgaagttaattgatgcagacaaatacgccgataagatcgacgaattaaacgcttttagaaagaaagcatttcaattcgcagtcgataatggttatgacattccagagtttactgattgggtataccctgatgttaaggttgatgaaacacaaatgttgtctgctactgctgctactgctggtgacaatgaataa

SEQ ID NO: 9 - DNA, 1719 bp, Escherichia coli
  source 1..1719: mol_type = other DNA
ttaccttagccagtttgttttcgccagttcgatcacttcatcaccgcgtccgctgatgattgcgcgcagcatatacaggctgaaacctttggcctgttcgagtttgatctgcggtggaatggctaactcttctttggcgaccaccacatccaccaacaccggaccgtcgatggagaaggcgcgttgcagggcttcatcaacttcagacgctttttctacacggatacccgtaatgccgcacgcttcggcaatgcgggcaaagtttgtgtcgtgtagttcggtgccgtcagtcaaatagccaccagctttcatctccatcgccacaaagcccagcacgctgttgttaaagacgacaattttcactggcagtttcatctgcactactgagaggaaatcgcccatcaacatgctaaaaccgccatcgccgcacatggcgaccacctgacgttctggctctgtcgcctgcgcacccagcgcctgcggcatggcgttagccatcgaaccgtggttaaacgaacctaacaggcgacgcttgccgttcatttttagataacgtgccgcccacaccgttggcgtaccaacgtcacaggtgaaaatagcgtcatcggcggcaaaatgactaatttgctgcgccagatattgcgggtgaatggctttctcgctcggtttagctaaatcgtccagccctttgcgggcgtcgcggtaatcttccagcgctttatccagaaacttgcgatcggctttttcttccaccaatggaagcaatgcacgcagagtcgacttgatatcgccgaccagtgccatatccaccttgctgtgagcgccgatgctggctgggttgatatcaatctgaatgattttggcatcggtcgggtagaaggcgcggtagggaaattgcgtgccgagtagcactaacgtgtcggcgttcatcatggtatggaaacctgacgagaagccgattaacccggtcattccaacatcatacggattatcgtattcgacatgttctttaccgcgcagggcatgaacaataggcgctttaattttcccggcaaactcaactaactctttatgcgcccccgcgcagccgctgccacacatcagggcgatattgctggaataacgcagcagttgcgccagtttgcgtaactcttcttcttccggcgtcacgactggttgtggcgcatgataccagtgcatggttgccccttctggcgcaggttttaacgccacgtcgcctggtaacacgacaaccgaaacgccacggttaagcaccgctttgcgcatggcaatcgccagtacttgtgggatctgctccgggctggaaaccagctcgcaatagtgactacattcgcggaatagctcttgtgggtgggtttcctggaaatagccgctgccaatttcgctggagggaatatgagcggcaatcgccagtaccggaacgtgattgcggtggcaatcgaacaggccgttgattaagtgcaggttgccggggccgcacgatccggcgcagaccgccagttctccgctaagttgtgcttcagcgccagcggcaaaggccgccacttcttcgtggcgggtggacatccactcgatggtgcccatgcgattaagactgtcactaagaccgttcagagagtcgcctgtgactccccagatgcgtttcacccctgccgattcgagtgttttggcgatataagctgcaaccgtttgtttcat

SEQ ID NO: 10 - DNA, 1374 bp, Escherichia coli
  source 1..1374: mol_type = other DNA
ttaatgcgcttttacggctttggcggttttctctttaaacagatagccgatacctaacacgatcagccataccgggatcaggtataccgaaatcgccattcctggggtcatcagcataatcaccagtaccgccgccataaacagcaggcagatccagttacccagcggataaagcagagcagggaagcgagttaccacgccttgttcctgcttggcgcgacggaatttcatatgcgccaggctaatcatcgcccagttgattaccagtgcagataccaccagcgccattaacagtccgaaagcggactctggggcaaggtagttaatcagtacgcacaacgccgttaccagtgcagacaccagaatggtatttactggtacaccacgtttatcgacagacgccagcgcttttggcgcattaccctgttgtgccagaccaaacagcatacggctgttgcaatatacgcagctgttgtacacggagagcgccgcagtcagtaccacgatgttcagcgcattcgccacaaaggtatcgcctaactcgtggaagatcagcacaaacggactggtatcggcggtaacgcgggtccacggcatcagtgagagcagaacggctaacgaaccaatatagaaaatcaggatgcggtagataacctggttagttgctttcggtatactttgctccgggttatcagcttctgctgcggtgatccccaccagttccagaccaccgaacgagaacatgataatcgccatcatcatcaccagcccggtgaagccgtgcggcaggaaaccaccctgatcccacaggttgctaacggtcgcctgcgggccgccgttgccactgaatagcagccagccgccgaagatgatcatcgctaccaccgcgataactttgataatggcaaaccagaactccatctcgccaaacactttaacgttggtcaggttgatggcgttaatcaccacaaagaatacggcggcagaaacccaggtggggatttccggataccagaactgaatgtatttacccacggcagtcagctcagccatggcaactaaaacgtacagtacccagtagttccagccagaggcgaaaccggcaaaactgccccagtatttataagcaaagtggctaaaggagcctgcgacaggttcttcgaccaccatttcacccagctgacgcatgatcagaaaggcgataaaaccagcaatggcgtaacccaggataatccctggccctgcggactgtattacggaggcgctacccaggaataacccggtccctatcgcgccacccagcgcgataagctgaatatggcggtttttaaggccgcgctttagctgctcgccgtgctgttgaccttccatcat

SEQ ID NO: 11 - DNA, 1212 bp, Escherichia coli
  source 1..1212: mol_type = other DNA
gtgaaaaacagaaccctgggaagtgtttttatcgtggcgggaaccacaattggcgcaggcatgctggcaatgccgctggctgcggccggtgttggttttagcgttacgttaatcttgttgattgggctttgggcgttgatgtgctacacggcgctattactgctggaggtgtaccagcatgttccggcagataccggtctgggcacgctggcaaaacgctatctgggacgctacggtcaatggctgacgggcttcagtatgatgttcttaatgtatgctctgactgcggcatacatcagcggtgccggtgaattgttggcctccagcatcagcgactggacaggtatttctatgtcggcaaccgctggcgtgctgttgttcacttttgttgccggtggcgtggtttgtgtcggaacttcactggtcgatttatttaaccgttttctgttcagcgccaagattatttttctggtggtaatgctggtactactgctgccgcatattcacaaagtgaatcttttaaccctgccgttgcaacaggggctggctctgtctgcaatcccggtgatttttacgtcgtttggttttcacggtagcgtgccgagtattgtcagctatatggatggcaacattcgtaagctacgctgggtgtttataatcggtagtgcgatccccctggtggcatatattttctggcaggtggcgacgcttggcagcattgattcaacaacctttatgggattgctggctaatcatgctggattaaacgggctgttacaggcgttacgcgaaatggtggcctctccgcatgttgagctggcagtgcatttatttgctgatttagccctcgccacgtcatttctcggcgttgcgttaggcttatttgattatctggctgatttatttcagcgttcaaataccgttggtggacggttgcaaactggtgcaattacgtttctgccgccgttggcgtttgcactgttttatccacgaggatttgtgatggcgctgggttacgccggtgtggcgctggcggtactggcattgattatcccttcgctgttgacctggcaaagcagaaagcacaatcctcaggcgggttaccgggtcaaaggtggtcgtccggcgctggtggtggtgtttctctgtggtattgctgtgattggcgtgcaatttttgattgcggcagggttgttaccagaagtggggtga

SEQ ID NO: 12 - DNA, 882 bp, Escherichia coli
  source 1..882: mol_type = other DNA
ttaaccacgacgtgtcgccagccagcagagcagggaaccgccgcagaccatcagcgcgccttgccagaacgagaacgacagcggggcgctgagcagcacggctgcaagcgctgaggaaagtacaggcgtaaaatacgaacctaccgccataatggtgacattgccatgcaatataccgacattccatgcagcataagcaaatcctaaggtaaatgccgcagagatgagtttaatcatgacgggcgtgctaaatatcatttctggttgtggcgtaagaaaatagtaaacccacagacttgctcccgttagcaggacaaaaacggtaattccattaaatccgcgtgcgtatttattcgttactgtgcaataggctgcccagataaacgcaccaatgaacgccaggaaataactcaatgggctggtggtgatattattgatgatttcatcataatgtaacccattgtcaccgcctaacacccaacagacgccgacgagggctaataataatccaggtacaatcaaccagttggttttctgaccattaaacagaatggcaaagagaattgtcaggctgggccacagatagttcaccatacccacttcaatcgcctgatgatgggtcgccgcataccctaaggaaagcgccagacagatttcatagctgacgaataacagactcccggcgagtaaatagcctttcgggatttgccgaatacgcggaaatccaaccgtgaagattaacagcagcccgcttaatgaatagatagcagctgcgccgccgaccgggccgagcccctcactgacaccgcgaatcaatcctaccatcgtgctccacaggacgatcgctatcagccctatgagcgttgctttttgtcgtgtcat

SEQ ID NO: 13 - DNA, 24 bp, synthetic construct
  source 1..24: mol_type = other DNA
cgtctcgccaaactggaaaaatgg

SEQ ID NO: 14 - DNA, 30 bp, synthetic construct
  source 1..30: mol_type = other DNA
cttttcaccccgatttgggaggccttattg

SEQ ID NO: 15 - DNA, 44 bp, synthetic construct
  source 1..44: mol_type = other DNA
ctcccaaatcggggtgaaaaggtgccggatgatgtgaatcatcc

SEQ ID NO: 16 - DNA, 29 bp, synthetic construct
  source 1..29: mol_type = other DNA
caatggtttctggagcaaattcaggtctg

SEQ ID NO: 17 - DNA, 58 bp, synthetic construct
  source 1..58: mol_type = other DNA
atataccgaaagtacgtctggttttagagctagaaatagcaagttaaaataaggctag

SEQ ID NO: 18 - DNA, 51 bp, synthetic construct
  source 1..51: mol_type = other DNA
cagacgtactttcggtatatactagtattatacctaggactgagctagctg

SEQ ID NO: 19 - DNA, 27 bp, synthetic construct
  source 1..27: mol_type = other DNA
ccagcaaacaaatggaaattactccgg

SEQ ID NO: 20 - DNA, 24 bp, synthetic construct
  source 1..24: mol_type = other DNA
gtctggtgtgatggacgtaaaccg

SEQ ID NO: 21 - DNA, 24 bp, synthetic construct
  source 1..24: mol_type = other DNA
ccaggcgttcaattaaggtttgcg

SEQ ID NO: 22 - DNA, 35 bp, synthetic construct
  source 1..35: mol_type = other DNA
gtttttatctcgccgaactgcgtcacgatcttgac

SEQ ID NO: 23 - DNA, 38 bp, synthetic construct
  source 1..38: mol_type = other DNA
gacgcagttcggcgagataaaaacagaaatcagggcag

SEQ ID NO: 24 - DNA, 23 bp, synthetic construct
  source 1..23: mol_type = other DNA
cgactctcgaactgctaacctgc

SEQ ID NO: 25 - DNA, 54 bp, synthetic construct
  source 1..54: mol_type = other DNA
attgccggaacacgcccacggttttagagctagaaatagcaagttaaaataagg

SEQ ID NO: 26 - DNA, 51 bp, synthetic construct
  source 1..51: mol_type = other DNA
cgtgggcgtgttccggcaatactagtattatacctaggactgagctagctg

SEQ ID NO: 27 - DNA, 21 bp, synthetic construct
  source 1..21: mol_type = other DNA
ccaggagaaagcatcagcacc

SEQ ID NO: 28 - DNA, 20 bp, synthetic construct
  source 1..20: mol_type = other DNA
gcaatcagatacccagcccg

SEQ ID NO: 29 - DNA, 24 bp, synthetic construct
  source 1..24: mol_type = other DNA
cggaatcaacgttgatgattgcgg

SEQ ID NO: 30 - DNA, 33 bp, synthetic construct
  source 1..33: mol_type = other DNA
aggcatattcgcacttcggcgtaaagatatccg

SEQ ID NO: 31 - DNA, 34 bp, synthetic construct
  source 1..34: mol_type = other DNA
gccgaagtgcgaatatgcctgatggtgcaacacc

SEQ ID NO: 32 - DNA, 22 bp, synthetic construct
  source 1..22: mol_type = other DNA
gatctgtctgacgtcaccctcg

SEQ ID NO: 33 - DNA, 53 bp, synthetic construct
  source 1..53: mol_type = other DNA
ccacgggacagtacgcacatgttttagagctagaaatagcaagttaaaataag

SEQ ID NO: 34 - DNA, 51 bp, synthetic construct
  source 1..51: mol_type = other DNA
atgtgcgtactgtcccgtggactagtattatacctaggactgagctagctg

SEQ ID NO: 35 - DNA, 24 bp, synthetic construct
  source 1..24: mol_type = other DNA
gtccagccagttttagatgcccag

SEQ ID NO: 36 - DNA, 23 bp, synthetic construct
  source 1..23: mol_type = other DNA
gttacagtcgccaattccatccc

SEQ ID NO: 37 - DNA, 28 bp, synthetic construct
  source 1..28: mol_type = other DNA
gaagaaataaccggcgttcagcctgtgc

SEQ ID NO: 38 - DNA, 41 bp, synthetic construct
  source 1..41: mol_type = other DNA
cgttctgataattcatgatctttagctgtcttggtttgccc

SEQ ID NO: 39 - DNA, 30 bp, synthetic construct
  source 1..30: mol_type = other DNA
atgaattatcagaacgacgatttacgcatc

SEQ ID NO: 40 - DNA, 44 bp, synthetic construct
  source 1..44: mol_type = other DNA
gtgaggacatggtatatctccttttacccgcgacgcgcttttac

SEQ ID NO: 41 - DNA, 43 bp, synthetic construct
  source 1..43: mol_type = other DNA
gggtaaaaggagatataccatgtcctcacgtaaagagcttgcc

SEQ ID NO: 42 - DNA, 36 bp, synthetic construct
  source 1..36: mol_type = other DNA
gtaggtgagttacagcagttcttttgctttcgcaac

SEQ ID NO: 43 - DNA, 40 bp, synthetic construct
  source 1..40: mol_type = other DNA
gcaaaagaactgctgtaactcacctaccaaacaatgcccc

SEQ ID NO: 44 - DNA, 30 bp, synthetic construct
  source 1..30: mol_type = other DNA
gcaaccattggatcccaatgcttcgtttcg

SEQ ID NO: 45 - DNA, 32 bp, synthetic construct
  source 1..32: mol_type = other DNA
ggatccaatggttgctgaattgaccgcattac

SEQ ID NO: 46 - DNA, 44 bp, synthetic construct
  source 1..44: mol_type = other DNA
gttggacatggtatatctccttttactggcgattgtcattcgcc

SEQ ID NO: 47 - DNA, 43 bp, synthetic construct
  source 1..43: mol_type = other DNA
gccagtaaaaggagatataccatgtccaacaatggctcgtcac

SEQ ID NO: 48 - DNA, 42 bp, synthetic construct
  source 1..42: mol_type = other DNA
ggctgaacgccggttatttcttcagttcagccaggcttaacc

SEQ ID NO: 49 - DNA, 44 bp, synthetic construct
  source 1..44: mol_type = other DNA
ggtaaaaggagatataccatgactaaccctgtaatcggtactcc

SEQ ID NO: 50 - DNA, 41 bp, synthetic construct
  source 1..41: mol_type = other DNA
gtttggtaggtgagttattcattgtcaccagcagtagcagc

SEQ ID NO: 51 - DNA, 37 bp, synthetic construct
  source 1..37: mol_type = other DNA
ggtgacaatgaataactcacctaccaaacaatgcccc

SEQ ID NO: 52 - DNA, 54 bp, synthetic construct
  source 1..54: mol_type = other DNA
cagggttagtcatggtatatctccttttacccgcgacgcgcttttactgcattc

SEQ ID NO: 53 - DNA, 76 bp, synthetic construct
  source 1..76: mol_type = other DNA
ttgacagctagctcagtcctaggtataatactagtaaagaggagaaaaagcttatgtcctcacgtaaagagcttgc

SEQ ID NO: 54 - DNA, 27 bp, synthetic construct
  source 1..27: mol_type = other DNA
ttacagcagttcttttgctttcgcaac

SEQ ID NO: 55 - DNA, 25 bp, synthetic construct
  source 1..25: mol_type = other DNA
gtgatatcgccaataccggattacg

SEQ ID NO: 56 - DNA, 40 bp, synthetic construct
  source 1..40: mol_type = other DNA
ggactgagctagctgtcaatgcggtgagttcaggttccgg

SEQ ID NO: 57 - DNA, 45 bp, synthetic construct
  source 1..45: mol_type = other DNA
gcaaaagaactgctgtaacaggcgttctacataaaacgcttacgc

SEQ ID NO: 58 - DNA, 23 bp, synthetic construct
  source 1..23: mol_type = other DNA
ggcgatgtgttgtgtgtaattgg

SEQ ID NO: 59 - DNA, 54 bp, synthetic construct
  source 1..54: mol_type = other DNA
ggcgaagaatatcatccatggttttagagctagaaatagcaagttaaaataagg

SEQ ID NO: 60 - DNA, 51 bp, synthetic construct
  source 1..51: mol_type = other DNA
catggatgatattcttcgccactagtattatacctaggactgagctagctg

SEQ ID NO: 61 - DNA, 21 bp, synthetic construct
  source 1..21: mol_type = other DNA
cgcattttgactgggttcggc

SEQ ID NO: 62 - DNA, 22 bp, synthetic construct
  source 1..22: mol_type = other DNA
gcggcactgtttcgtgataacc

SEQ ID NO: 63 - DNA, 75 bp, synthetic construct
  source 1..75: mol_type = other DNA
ttgacagctagctcagtcctaggtataatactagtaaagaggagaaaaagcttatgtccaacaatggctcgtcac

SEQ ID NO: 64 - DNA, 39 bp, synthetic construct
  source 1..39: mol_type = other DNA
gattgagagttttatttcttcagttcagccaggcttaac

SEQ ID NO: 65 - DNA, 25 bp, synthetic construct
  source 1..25: mol_type = other DNA
gtatctcatcgagaacttgcctgcc

SEQ ID NO: 66 - DNA, 39 bp, synthetic construct
  source 1..39: mol_type = other DNA
gactgagctagctgtcaaaccgttccagagagggggacc

SEQ ID NO: 67 - DNA, 35 bp, synthetic construct
  source 1..35: mol_type = other DNA
gaagaaataaaactctcaatctgatcggttcctgc

SEQ ID NO: 68 - DNA, 21 bp, synthetic construct
  source 1..21: mol_type = other DNA
gtgcggattaaatcccgcgac

SEQ ID NO: 69 - DNA, 54 bp, synthetic construct
  source 1..54: mol_type = other DNA
ggtgaaaacgactatcacgggttttagagctagaaatagcaagttaaaataagg

SEQ ID NO: 70 - DNA, 51 bp, synthetic construct
  source 1..51: mol_type = other DNA
ccgtgatagtcgttttcaccactagtattatacctaggactgagctagctg

SEQ ID NO: 71 - DNA, 23 bp, synthetic construct
  source 1..23: mol_type = other DNA
cggatacaatgaccagttcctgg

SEQ ID NO: 72 - DNA, 19 bp, synthetic construct
  source 1..19: mol_type = other DNA
cggtttccagtgccacgtc

SEQ ID NO: 73 - DNA, 21 bp, synthetic construct
  source 1..21: mol_type = other DNA
ggcaacactttgccgttgtgg

SEQ ID NO: 74 - DNA, 32 bp, synthetic construct
  source 1..32: mol_type = other DNA
cataatcgccgaactggcgaaaacaaactggc

SEQ ID NO: 75 - DNA, 34 bp, synthetic construct
  source 1..34: mol_type = other DNA
tcgccagttcggcgattatgcgagaaccaaatcc

SEQ ID NO: 76 - DNA, 23 bp, synthetic construct
  source 1..23: mol_type = other DNA
cgatgagtggcgtaactatccgg

SEQ ID NO: 77 - DNA, 54 bp, synthetic construct
  source 1..54: mol_type = other DNA
ggtgaaaatagcgtcatcgggttttagagctagaaatagcaagttaaaataagg

SEQ ID NO: 78 - DNA, 51 bp, synthetic construct
  source 1..51: mol_type = other DNA
ccgatgacgctattttcaccactagtattatacctaggactgagctagctg

SEQ ID NO: 79 - DNA, 21 bp, synthetic construct
  source 1..21: mol_type = other DNA
gtcaacatgcagcgccagatt

SEQ ID NO: 80 - DNA, 21 bp, synthetic construct
  source 1..21: mol_type = other DNA
gatctacaacgtgcgtacgcc

SEQ ID NO: 81 - DNA, 22 bp, synthetic construct
  source 1..22: mol_type = other DNA
cgtgagtatttgcgtgagctgc

SEQ ID NO: 82 - DNA, 30 bp, synthetic construct
  source 1..30: mol_type = other DNA
acgaggtttctctctctacgccctcacccg

SEQ ID NO: 83 - DNA, 30 bp, synthetic construct
  source 1..30: mol_type = other DNA
cgtagagagagaaacctcgtgcggtggttg

SEQ ID NO: 84 - DNA, 27 bp, synthetic construct
  source 1..27: mol_type = other DNA
ggtcttaccaatttcatgtctgtgacg

SEQ ID NO: 85 - DNA, 54 bp, synthetic construct
  source 1..54: mol_type = other DNA
aatcaccacaaagaatacgggttttagagctagaaatagcaagttaaaataagg

SEQ ID NO: 86 - DNA, 51 bp, synthetic construct
  source 1..51: mol_type = other DNA
cagctagctcagtcctaggtataatactagtaatcaccacaaagaatacgg

SEQ ID NO: 87 - DNA, 20 bp, synthetic construct
  source 1..20: mol_type = other DNA
ttggcgcaggtaaagttcgt

SEQ ID NO: 88 - DNA, 20 bp, synthetic construct
  source 1..20: mol_type = other DNA
tgttttgccagttcgcgttc

SEQ ID NO: 89 - DNA, 25 bp, synthetic construct
  source 1..25: mol_type = other DNA
gcggcgaaggtctgtattttatcga

SEQ ID NO: 90 - DNA, 38 bp, synthetic construct
  source 1..38: mol_type = other DNA
ctatctgagctttcttctgtcctgacgatctttatgag

SEQ ID NO: 91 - DNA, 49 bp, synthetic construct
  source 1..49: mol_type = other DNA
cgtcaggacagaagaaagctcagatagcctcaaattccttattgggtgc

SEQ ID NO: 92 - DNA, 26 bp, synthetic construct
  source 1..26: mol_type = other DNA
ctggcttttcaacatatggccgatac

SEQ ID NO: 93 - DNA, 54 bp, synthetic construct
  source 1..54: mol_type = other DNA
agaaaatatatgccaccagggttttagagctagaaatagcaagttaaaataagg

SEQ ID NO: 94 - DNA, 51 bp, synthetic construct
  source 1..51: mol_type = other DNA
cagctagctcagtcctaggtataatactagtagaaaatatatgccaccagg

SEQ ID NO: 95 - DNA, 27 bp, synthetic construct
  source 1..27: mol_type = other DNA
gagcagcatgaagaagagaaactgttc

SEQ ID NO: 96 - DNA, 23 bp, synthetic construct
  source 1..23: mol_type = other DNA
ggcgtcagagaaagagatgacgc

SEQ ID NO: 97 - DNA, 40 bp, synthetic construct
  source 1..40: mol_type = other DNA
ggtaaaaggagatataccatgacacgacaaaaagcaacgc

SEQ ID NO: 98 - DNA, 35 bp, synthetic construct
  source 1..35: mol_type = other DNA
gtttggtaggtgagttaaccacgacgtgtcgccag

SEQ ID NO: 99 - DNA, 34 bp, synthetic construct
  source 1..34: mol_type = other DNA
cgtcgtggttaactcacctaccaaacaatgcccc

SEQ ID NO: 100 - DNA, 50 bp, synthetic construct
  source 1..50: mol_type = other DNA
ctttttgtcgtgtcatggtatatctccttttacccgcgacgcgcttttac

SEQ ID NO: 101 - DNA, 53 bp, synthetic construct
  source 1..53: mol_type = other DNA
ttgacagctagctcagtcctaggtataatactagtaaagaggagaaaaagctt